(19) United States
(12) Patent Application Publication
(10) Pub. No.: US 2005/0064400 A1
(43) Pub. Date: Mar. 24, 2005

(54) System and method for temperature gradient capillary electrophoresis

(76) Inventors: Zhiyong Guo, State College, PA (US); Zhaowei Liu, Port Matilda, PA (US); Qingbo Li, State College, PA (US)

Correspondence Address: FISH & RICHARDSON PC, 225 FRANKLIN ST, BOSTON, MA, 02110, US

(21) Appl. No.: 10/287,808

(22) Filed: Nov. 5, 2002

Related U.S. Application Data
(63) Continuation of application No. PCT/US02/33215, filed on Oct. 18, 2002.
(60) Provisional application No. 60/329,739, filed on Oct. 18, 2001.

(51) Int. Cl. C12Q 1/68; G06F 19/00; G01N 33/48; G01N 33/50

(52) U.S. Cl. 435/6; 702/20

(57) ABSTRACT

The present invention relates to a method for determining the presence of a mutation in a first sample comprising first polynucleotides. The reference sample comprises reference polynucleotides. The first sample and a reference sample are subjected to electrophoresis in the presence of at least one intercalating dye. During electrophoresis the temperature of the first sample and the reference sample is changed by an amount sufficient to change an electrophoretic mobility of at least one of the first or reference polynucleotides. Fluorescence intensity data are obtained. The fluorescence intensity data are indicative of the presence of the first and reference polynucleotides. The data are processed to determine the presence of mutation in the first polynucleotides.

### RELATED APPLICATIONS

The present application is a continuation of PCT Application No. US02/33215, filed in the United States Oct. 18, 2002, and claims the benefit of U.S. Provisional Application No. 60/329,739, filed Oct. 18, 2001; the present application is related to U.S. application Ser. No. ______, filed Nov. 5, 2002, and having attorney docket number 9046-046-999. Each of the foregoing applications is incorporated herein.

### FIELD OF THE INVENTION

The invention relates to a system and method for separating materials having temperature-dependent electrokinetic mobilities. More particularly, the invention relates to time-dependent temperature gradient electrokinetic separation of materials including DNA fragments.

### BACKGROUND

Detection of mutations and variations occurring in DNA has become increasingly important in the fields of genetics, molecular diagnostics and cancer research. One type of variation, single-nucleotide polymorphism (SNP), has attracted much attention because it is the most common form of genetic variation. This type of single-base substitution in the genome occurs at a frequency of >1% in the human population. A recent estimate is that there is about one SNP per 1000 bp in human DNA. Other types of mutations involve insertion and deletion, and are found to occur at about one per 12 kb. The determination of SNPs can be used to study genetic linkages and for the diagnosis of diseases, especially cancer.

One way to fully characterize a mutation is to perform DNA sequencing on the sample. However, current DNA sequencing techniques are laborious and expensive. Large-scale DNA sequencing to detect mutations is also not efficient because a large portion of the sequences will give negative results considering that mutation is the exception. To save time and cost, rapid screening methods need to be developed to identify both known point mutations and unknown point mutations before any further characterization is undertaken.

The detection of mutation can be accomplished by using oligonucleotide arrays or DNA chips. Even though the number of analysis sites that can be packed into a small area array is very large, one must use multiple spots to span each mismatch (mutation). Using arrays, the match/mismatch discrimination is not entirely definitive, since different sequences have different melting temperatures. Ideally, one would have slightly different temperatures at each site within the array of sites. The other issue is time. In a representative mode of operation, the DNA is applied to the array and hybridization is carried out at 44° C. for 15 h at 40 rpm. The array of sites is then washed and stained before imaging. A third issue is that the DNA arrays are presently quite costly if one wants to span all possible mutations and probe scores of clinical samples at a time. Clearly, further development is needed to speed up the process and to make it more cost effective.

Mutations in DNA are readily detected by mass changes, such as by mass spectroscopic techniques. Substitutions are not so obvious because of the limited mass resolution of instruments that are reasonably accessible at present. Positional switches will not be detected at all because these do not result in a mass change.

A popular clectrophoresis method to detect polymorphism is to rely on slight changes in conformations in single-stranded DNA (SSCP). This technique relies on subtle electrophoretic mobility differences between single strands of DNA that have different sequences. The mobility differences arise because, under the proper conditions, the different strands will have subtly different conformations in the separation medium. There are at least three important limitations to the sensitivity of SSCP analysis. First, the “mildly” denaturing condition is not well defined and may have to be optimized for each DNA region. This is because the conformation of each strand, and therefore any changes in conformation, is specific to a particular sequence. Therefore, the mobility differences will not be observed if the separation conditions are not optimized for each particular sequence in the sample. Second, visualization after the separation is complex. For example, the introduction of a radionucleotide probe or a fluorescence label into the DNA strand requires prior knowledge of the specific sequences of DNA regions around the point of mutation. Third, at present the assay is not reliable with fragments greater than around 200 bp and the sensitivity is only 60-95%.

For the analysis of double-stranded DNA, conformation-sensitive gel electrophoresis (CSGE) is possible. This approach is based on slight differences in conformations between the homoduplex and the heteroduplex DNA fragments. Just as discussed for SSCP above, the optimal gel and buffer conditions are particular to each sequence. Only when applied together with SSCP can the mutation detection rate approach 100%.

A different approach is to use denaturing gradient gel electrophoresis (DGGE). Separation is performed at a constant temperature but with a gel constructed to provide various degrees of denaturation along its length. If the sequence is known around the region probed, the mutation detection rate can reach 100%, but irreproducibility in creating identical gels makes implementation difficult. Also, it is often necessary to attach an artificial GC-rich sequence to the respective ends of the two strands to provide optimum separation.

Compared to SSCP or CSGE, DGGE can handle longer DNA fragments and is less time-consuming. An analog of DGGE is temperature-gradient gel electrophoresis (TGGE). In TGGE, instead of a denaturant gradient along the gel, a spatial or temporal temperature gradient is used to perform the same function. A simpler scheme is to apply constant denaturing capillary electrophoresis (CDCE). But this is again limited to defined mutations.

Capillary electrophoresis (CE) provides rapid analysis, a small sample requirement, and high sensitivity. It has been successfully used in many DNA analysis fields like sequencing and genotyping. Recently developed multiple-capillary arrays are ideal for high-throughput analysis. It is possible to detect mutations using CDCE with laser-induced fluorescence of covalent tags or with DGCE using a secondary polymer concentration gradient to refocus the sample band in addition to a denaturant gradient. The construction of gradients in the above techniques are tedious and hard to reproduce, especially for a capillary array.

The temperature of the separation medium within a capillary can be modified internally through ohmic heating by varying the electric potential across the capillary. Limitations of this technique include the narrow temperature range that can be achieved and the mutual dependence of the temperature and the electric field. This dependence is undesirable because the optimal separation conditions for a particular sample may not be achieved at an electric field consistent with heating the capillary to the required temperature.

### SUMMARY OF THE INVENTION

One aspect of the present invention relates to a system for determining the presence of a mutation in at least one sample polynucleotide. In one embodiment, the system comprises a processor configured to at least receive (1) at least a first set of sample electrophoresis data indicative of a temperature gradient electrophoresis (TGE) separation of at least one sample polynucleotide, and (2) at least a first set of reference electrophoresis data indicative of a TGE separation of at least one reference polynucleotide, and process at least a subset of the first set of sample electrophoresis data and at least a subset of the first set of reference electrophoresis data to prepare a set of result data corresponding to the first set of sample electrophoresis data, wherein the set of result data includes data indicative of whether a sample polynucleotide of the first set of sample electrophoresis data includes a mutation.

The system may further include a display and the processor be configured to display result data corresponding to the first set of sample electrophoresis data. The processor may be configured to write result data to a storage medium.

The displayed result data includes at least one indicium of whether a sample polynucleotide of the first set of sample electrophoresis data includes a mutation. Result data corresponding to the first set of sample electrophoresis data may include an indicium of whether the first set of sample electrophoresis data includes more than one sample polynucleotide. Displayed result data corresponding to the first set of sample electrophoresis data may include an indicium corresponding to each sample polynucleotide of the first set of sample electrophoresis data, each indicium being indicative of whether a respective one of the sample polynucleotides includes a mutation.

The processor may be configured to at least receive at least a second set of sample electrophoresis data, process at least a subset of the second set of sample electrophoresis data to prepare a set of result data corresponding to the second set of sample electrophoresis data, and display result data corresponding to the second set of sample electrophoresis data, the displayed result data including an indicium of whether a sample polynucleotide of the second set of sample electrophoresis data includes a mutation.

The processor may be configured to at least receive at least a second set of reference electrophoresis data receive user input defining members of a first group of sets of sample electrophoresis for processing with respect to the first set of reference electrophoresis data, the first group of sets including the first set of sample electrophoresis data receive user input defining members of a second group of sets of sample electrophoresis for processing with respect to the second set of reference electrophoresis data process at least a subset of each member of the first group and at least a subset of the first set of reference electrophoresis data to prepare respective sets of result data corresponding to members of the first group, and process at least a subset of each member of the second group and at least a subset of the second set of reference electrophoresis data to prepare respective sets of result data corresponding to members of the second group. The system may include a display and the processor be configured to simultaneously display result data corresponding to members of the first and second groups of sets of sample electrophoresis data. The displayed result data may include a plurality of indicia, each of the indicia indicative of whether a sample polynucleotide of a respective one of the sets of sample electrophoresis data includes a mutation.

The processor may be configured to normalize at least one of the subset of the first set of sample electrophoresis data and the subset of the first reference electrophoresis data and to process the normalized data.

The processor may be configured to receive user input indicative of a size of the subset of the first set of sample electrophoresis data and a size of the subset of the first set of reference electrophoresis data and to process the subsets of electrophoresis data to prepare the result data corresponding to the first set of sample electrophoresis data.

The system may further comprise a capillary array electrophoresis system configured to obtain sample electrophoresis data and reference electrophoresis data.

Another aspect of the invention relates to a computer-readable medium comprising executable software code, the code for determining the presence of a mutation in at least one sample polynucleotide. In one embodiment the computer-readable medium comprises code to receive (1) at least a first set of sample electrophoresis data indicative of a temperature gradient electrophoresis (TGE) separation of at least one sample polynucleotide, and (2) at least a first set of reference electrophoresis data indicative of a TGE separation of at least one reference polynucleotide, and code to process at least a subset of the first set of sample electrophoresis data and at least a subset of the first set of reference electrophoresis data to prepare a set of result data corresponding to the first set of sample electrophoresis data, wherein the set of result data includes data indicative of whether a sample polynucleotide of the first set of sample electrophoresis data includes a mutation.

The code may include code to display result data corresponding to the first set of sample electrophoresis data. The code may include code to display at least one indicium of whether a sample polynucleotide of the first set of sample electrophoresis data includes a mutation. The display may include an indicium of whether the first set of sample electrophoresis data includes more than one sample polynucleotide. The code may include code to display an indicium corresponding to each sample polynucleotide of the first set of sample electrophoresis data, each indicium being indicative of whether a respective one of the sample polynucleotides includes a mutation.

The code may include code to receive at least a second set of sample electrophoresis data, code to process at least a subset of the second set of sample electrophoresis data to prepare a set of result data corresponding to the second set of sample electrophoresis data, and code to display result data corresponding to the second set of sample electrophoresis data, the displayed result data including an indicium of whether a sample polynucleotide of the second set of sample electrophoresis data includes a mutation.

The code may include code to receive at least a second set of reference electrophoresis data, code to receive user input defining members of a first group of sets of sample electrophoresis for processing with respect to the first set of reference electrophoresis data, the first group of sets including the first set of sample electrophoresis data, code to receive user input defining members of a second group of sets of sample electrophoresis for processing with respect to the second set of reference electrophoresis data, code to process at least a subset of each member of the first group and at least a subset of the first set of reference electrophoresis data to prepare respective sets of result data corresponding to members of the first group, and code to process at least a subset of each member of the second group and at least a subset of the second set of reference electrophoresis data to prepare respective sets of result data corresponding to members of the second group.

The code may be configured to simultaneously display result data corresponding to members of the first and second groups of sets of sample electrophoresis data. The code may be configured to display a plurality of indicia, each of the indicia indicative of whether a sample polynucleotide of a respective one of the sets of sample electrophoresis data includes a mutation.

Another aspect of the present invention relates to a method for interacting with a computer to determine the presence of mutation in at least one sample polynucleotide. In one embodiment, the method comprises the steps of executing an application which includes one or more windows having one or more controls, manipulating at least one of said controls to define members of a group of sample electrophoresis data, manipulating at least one of said controls for defining a set of reference electrophoresis data to be used for processing members of the group, manipulating at least one of said controls to select a first subset of each member of the group and a first subset of the reference electrophoresis data, and manipulating at least one of said controls to execute code configured to process the first subsets of the members of the group and the first subset of the set of reference electrophoresis data to prepare result data corresponding to each member of the group, wherein the result data corresponding to each member of the group includes data indicative of whether a first sample polynucleotide of the corresponding sample electrophoresis data includes a mutation.

The method may include the further steps of manipulating at least one of said controls to select a second subset of each member of the group and a second subset of the reference electrophoresis data and manipulating at least one of said controls to execute code configured to process the second subsets of the members of the group and the second subset of the set of reference electrophoresis data to prepare result data corresponding to each member of the group, wherein the result data corresponding to each member of the group includes data indicative of whether a second sample polynucleotide of the corresponding sample electrophoresis data includes a mutation.

Another aspect of the invention relates to a method of processing electrophoresis data obtained by temperature gradient electrophoresis (TGE) separation to provide data indicative of the presence of a single polynucleotide polymorphism (SNP) or a mutation in a sample compound of a biological sample. In one embodiment, the method comprises providing reference electrophoresis data comprising a plurality of reference data points (drᵢ, mᵢ), the reference data points defining at least one reference peak and having been obtained by TGE of at least one reference compound, and where, for the ith reference data point, drᵢ is a detection value indicative of a detector signal and mᵢ is a migration coordinate, providing sample electrophoresis data comprising a plurality of sample data points (dsⱼ, mⱼ), the sample data points defining at least one sample peak and having been obtained by TGE of at least one sample compound, and where, for the jth sample data point, dsⱼ is a detection value indicative of a detector signal and mⱼ is a migration coordinate, normalizing the reference and sample electrophoresis data based on at least one detection value of the reference electrophoresis data and at least one detection value of the sample electrophoresis data, determining a plurality of detection value differences Δ between the reference and sample electrophoresis data, where the kth detection value difference Δₖ is indicative of a difference between (1) the detection value drᵢ of the reference electrophoresis data point having the migration coordinate mᵢ and (2) the detection value dsⱼ of the sample electrophoresis data point having the migration coordinate mⱼ, and wherein the plurality of detection value differences Δ are indicative of the presence of an SNP or mutation in the sample compound of the biological sample.

In another embodiment, the method includes the steps of providing reference electrophoresis data comprising a plurality of reference data points (drᵢ, mᵢ), the reference data points defining at least one reference peak and having been obtained by TGE of at least one reference compound, and where, for the ith reference data point, dsᵢ is a detection value indicative of a detector signal and mᵢ is a migration coordinate, providing sample electrophoresis data comprising a plurality of sample data points (sⱼ, mⱼ), the sample data points defining at least one sample peak and having been obtained by TGE of at least one sample compound, and where, for the jth sample data point, sⱼ is a detection value indicative of a detector signal and mⱼ is a migration coordinate, normalizing the reference and sample electrophoresis data based on at least one detection value of the reference electrophoresis data and at least one detection value of the sample electrophoresis data, and determining a covariance between the reference and sample electrophoresis data, wherein the covariance is indicative of the presence of an SNP or mutation in the sample compound of the biological sample.

Another embodiment of the present invention relates to an improved method for detecting mutations in a polynucleotide-containing sample by subjecting the sample polynucleotides to temperature gradient electrophoresis and obtaining spectroscopic intensity data indicative of the presence of the polynucleotides. Preferably, the sample comprises at least one pair of polynucleotide sequences. Each member polynucleotide sequence is preferably a double strand of DNA. A preferred pair of polynucleotides sequences comprises a heteroduplex DNA fragment and a homoduplex DNA fragment. The presence of a mutation is determined by comparing the spectroscopic intensity indicative of the presence of the member polynucleotides of a pair.

Yet another embodiment of the present invention relates to a temperature gradient electrophoresis-based method for generating data indicative of the presence of a single polynucleotide polymorphism or a mutation in a biological sample. The sample includes non-desalted polymerase chain reaction (PCR) products. Thus, the biological sample has preferably not been desalted.

### BRIEF DESCRIPTION OF THE DRAWINGS

The present invention is described in detail below with reference to the drawings in which:

FIG. 1a shows an embodiment of a mutation detection device having a gas cooled portion in accordance with the present invention;

FIG. 1b shows an embodiment of a mutation detection device having a thermoelectrically cooled portion in accordance with the present invention;

FIG. 1c shows an embodiment of a mutation detection device having a liquid cooled portion in accordance with the present invention;

FIG. 2 shows heteroduplex and homoduplex fragments, in accordance with the present invention;

FIGS. 3a and 3b show exemplary electrophoresis data of a sample and a reference polynucleotide-containing compound, respectively, in accordance with the present invention;

FIGS. 4a and 4b show exemplary electrophoresis data of a sample and a reference polynucleotide-containing compound, respectively, in accordance with the present invention;

FIG. 5 shows a flow chart including steps for performing mutation detection in accordance with the present invention;

FIGS. 6a-6c show an exemplary user interface by which a user may define groups of sample electrophoresis data in accordance with the present invention;

FIG. 7 shows an exemplary user interface by which a user may define a size of one or more subsets of electrophoresis data in accordance with the present invention; and

FIG. 8 shows an exemplary interface for presenting result data of mutation one or more mutation determinations in accordance with the present invention.

### DETAILED DESCRIPTION OF THE PREFERRED EMBODIMENT

The present invention relates to a rapid method of using capillary electrophoresis for determining the presence of a single polynucleotide polymorphism or mutation in a sample, which preferably comprises polynucleotides. As used herein, samples refer to samples that have components that are to be analyzed to determine the presence of a single polynucleotide polymorphism or mutation therein. In addition to polynucleotide containing compounds, such as DNA fragments, the present invention is adaptable to other compounds, such as proteins, peptides, RNA, and the like, having a temperature dependent mobility in the presence of an electric field.

It is understood in the art that a single polynucleotide polymorphism (SNP) is an inherited variation in the genome of an individual. Thus, a SNP can be detected by, for example, comparing DNA of one individual of a population with DNA of another individual of the population. A mutation, on the other hand, is a change in a genome sequence that results from a perturbation, such as exposure to radiation or a chemical mutagen. A mutation can be detected by, for example, comparing DNA of an individual before exposure of the individual to a perturbation with DNA of the individual after exposure of the individual to the perturbation. The present method is equally adapted to determining the presence of SNPs or mutations. As used herein, it should be understood that the term “mutation” is meant to include variations, such as deletions, insertions, or substitutions, in polynucleotide sequences whether those variations result from an external perturbation, such as a mutation, or are inherited, such as SNP's. Thus, the terms “mutation” and “single polynucleotide polymorphism” are used interchangeably herein.

In the method of the invention, a temporal temperature gradient is applied to a temperature controlled zone of an electrophoretic separation medium. During the temperature gradient, each component of the sample preferably experiences a first temperature where the component has a first mobility and a second temperature where the component has a second, different mobility. Of course, the first and second temperatures will likely be different for different sample components.

In one embodiment of the present invention, the sample components comprise polynucleotides. During electrophoresis, the polynucleotides experience a first temperature, at which temperature the polynucleotides are substantially annealed or unmelted, and a second temperature, at which temperature the polynucleotides become at least partially melted or denatured. As used herein, the terms “melt” and “melted” are respectively synonymous with the terms “thermally denature” and “thermally denatured.” During electrophoresis, the temperature of the temperature controlled zone preferably changes by at least about 1° C. and more preferably at least about 7.5° C. The temperature of the temperature controlled zone is set with a precision of better than about 0.5° C. for example better than about 0.1° C.

The presence of components of the sample and reference is preferably determined by obtaining spectroscopic data indicative of the presence of sample components. The spectroscopic data can include, for example, absorbance data or fluorescence data.

In one embodiment, the electrophoretic separation medium comprises an intercalating dye, such as ethidium bromide to allow fluorescence detection of the separated polynucleotides. The intercalating dye preferentially allows detection of double stranded DNA as compared to single stranded DNA. In one embodiment, the separation medium is substantially free of a covalent tag suitable for fluorescence detection of single strands of DNA and the separation medium is completely free of a covalent tag. By substantially free it is meant that the presence of any covalent tag suitable for fluorescence detection of single strands of DNA is insufficient to interfere with the detection of sample compounds using fluorescence resulting from the intercalating dye. In one embodiment, the polynucleotides to be separated are preferably substantially free of fluorescent dyes that covalently tag single stranded DNA. Multiple samples comprising polynucleotides, such as DNA fragments, can be simultaneously analyzed.

The electrophoresis data of the sample components is indicative of the presence of mutation in the sample components. By indicative, it is meant that the electrophoresis data of the sample components can be compared with electrophoresis data obtained from one or more references to determine the presence of mutation. The presence of mutations are preferably identified by comparing electrophoresis data resulting from a heteroduplex polynucleotide with electrophoresis data resulting from a homoduplex reference polynucleotide without prior knowledge of the sequence of the heteroduplex polynucleotide.

In another embodiment, the electrophoretic medium comprises a tagging agent, such as an intercalating tag, having an extinction coefficient that is sufficiently large to allow the presence of the sample components to be determined by detecting the absorbance of the tagging agent.

In another embodiment, the presence of the sample polynucleotides is determined by directly measuring the absorbance of the sample components themselves rather than by measuring the absorbance of a tagging agent.

The invention is suitable for high-throughput screening of mutations and single-nucleotide polymorphisms, by multiplexing large numbers of samples. Preferably, at least as many as 96 electrophoretic separations can be simultaneously performed.

Temperature Gradient Electrophoresis

Preferred electrophoretic mutation detection devices for performing mutation detection by temperature gradient electrophoresis in accordance with the present invention have a plurality of separation lanes, such as at least 48, for example, 96 separation lanes. An example of a suitable electrophoresis system is the model SCE9610 electrophoresis system available from Spectrumedix, LLC of State College, Pa. This system includes 96 capillaries, each of which may be used to subject at least one of one reference and at least one sample to temperature gradient electrophoresis.

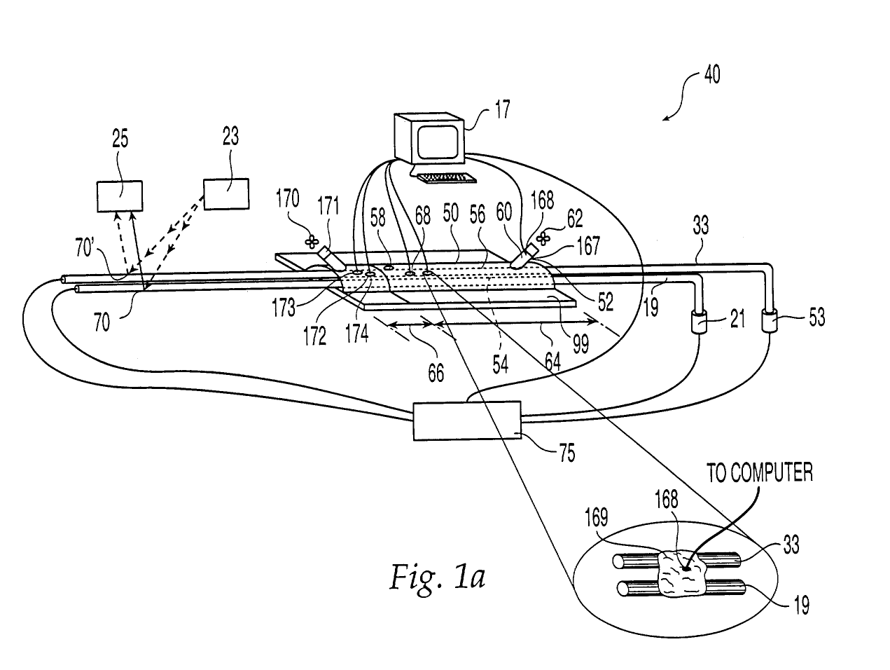

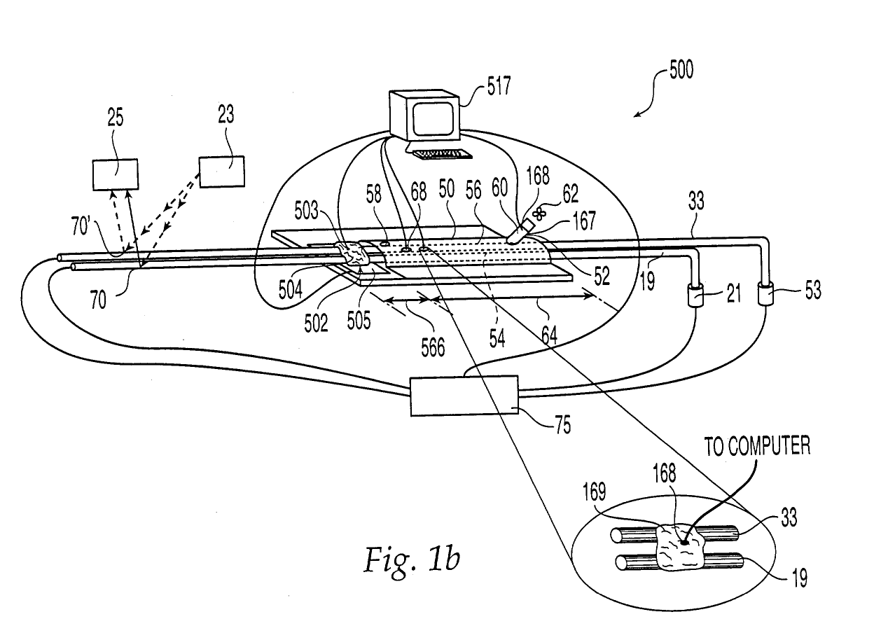

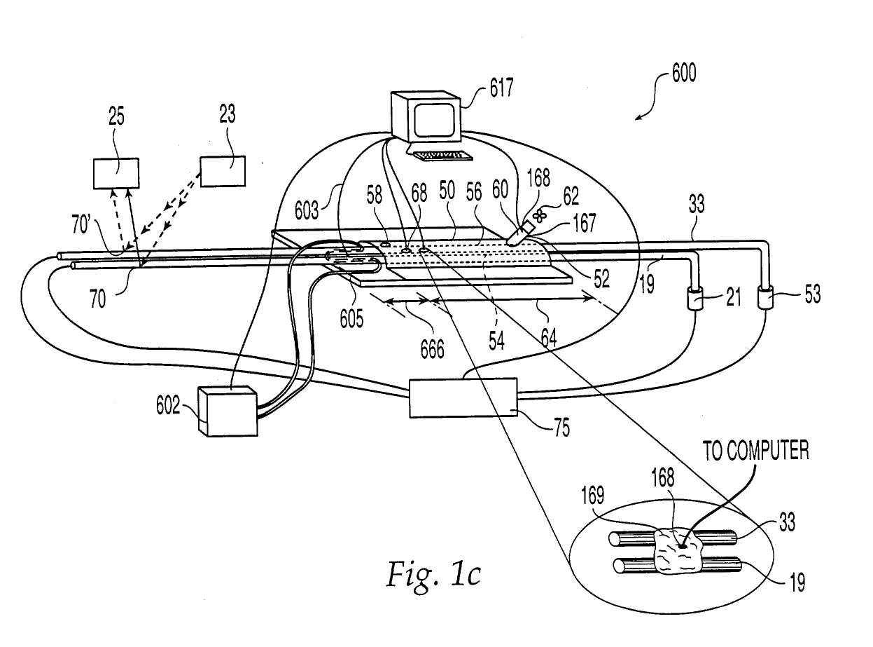

FIG. 1a shows an embodiment of the present electrophoretic mutation detection device 40. A separation lane, such as a sample capillary 33, is provided to electrophoretically separate unknown sample compounds. By separation lane, it is meant any structure configured and arranged to separate a sample using electrophoresis. Preferred structures include capillaries and microfabricated channels. The separation takes place within the internal bores of the capillaries or the interior of the microfabricated channels. As discussed below, the internal bore or interior of the channels are filled with a separation medium suitable for supporting an electrophoretic separation. Although the electrophoretic mutation detection device preferably includes more than two separation lanes, for clarity, only two are shown in FIGS. 1a-1c discussed herein.

Capillary 33 is arranged to be in fluid contact with a sample reservoir 53, which is configured to contain a volume of sample sufficient to perform an analysis. The sample is preferably suspended or dissolved in a buffer suitable for electrophoresis. Examples of suitable sample reservoirs include the wells of a microtitre plate, a vessel configured to perform PCR amplification of a volume of sample, a reservoir of a microfabricated lab on a chip device, and the like.

Mutation detection device 40 is preferably provided with an optional reference capillary 19 configured to simultaneously separate a reference sample comprising reference polynucleotides. Reference capillary 19 includes a reference reservoir 21 configured to contain the reference sample. Reference capillary 19 and reference reservoir 21 have the same characteristics as the sample capillary 33 and sample reservoir 53. An optional support 99 is provided to stabilize capillaries 19, 33.

Device 40 includes a power supply 75 for providing a voltage and current sufficient for electrophoretic separation of a sample. The power supply is preferably configured to allow at least one of the current or resistance of the capillary to be monitored during a separation. Preferably, the current or resistance data is received by the computing device 17 to allow the electric potential to be varied to maintain a constant current or resistance. This is discussed in more detail below.

A temperature control zone 50 of sample capillary 33 and optional reference capillary 19 are placed in thermal contact with an external heat source, such as a gas, which is used to heat portions of capillaries 33, 19. Air or nitrogen are examples of gas that can be used. Because the capillaries 33, 19 preferably have a radius of less than about 500 microns, the thermal conductivity between the separation medium within the internal bores of the capillaries and the gas is sufficiently high to allow the gas to heat the separation medium. Thus, during electrophoresis, the external heat source, rather than ohmic heating of the separation medium itself, is the dominant source of any substantial temperature changes or fluctuations within the separation medium within the capillary. Because sample components, such as polynucleotides, migrate within the separation medium, which typically contains a liquid, the sample components are also in thermal contact with the external heat source.

Temperature control zone 50 preferably extends for a length Ttemp 64 of the capillaries. At least one inlet port 52 is provided to introduce the heated gas to a heated region 54 between the capillaries and a thermal jacket 56. At least one outlet 58 is provided to allow the gas to exit from heated region 54. A fan 62 or other device to force the gas into the inlet and out of the exit is provided. Thermal jacket 56, which can entirely surround capillaries 33, 19, insulates temperature control zone 50 to reduce heat loss therefrom and to maintain the gas in contact with capillaries 33, 19.

The gas can be heated by, for example, passing the gas over a resistively heated filament 167 or a heat exchanger prior to introducing the gas into heated region 54. Filament 167 can be located within or adjacent inlet port to reduce heat loss that would occur if hot gas were transported from a location remote from device 40.

At least one temperature sensor 68 is preferably used to determine the temperature of the gas in contact with capillaries 33, 19 in the portion Tₜₑₘₚ. An additional temperature sensor 168 is placed in thermal contact with the capillaries in the portion Tₜₑₘₚ. Preferably, sensor 168 is embedded in a mass of thermally conductive material 169, so that the temperature reported by sensor 168 is indicative of the temperature within the internal bore of capillaries 33, 19. Suitable thermally conductive materials include, for example, the TCE series of thermal epoxies available from Melcor, Trenton, N.J.

A computer 17 receives signals from sensors 68, 168 indicative of the gas temperature, and capillary temperature, respectively. The temperature of filament 167 is preferably under control of computer 17, which is configured to vary the current flowing through the filament. During operation, computer 17 compares the temperature received from sensor 168 (capillary bore temperature) with a predetermined target temperature, which can vary as a function of time. If the capillary bore temperature is less than the target temperature, computer 17 raises the temperature of filament 167, such by increasing the amount of current flowing through filament 167, to increase the gas temperature in contact with capillaries 33, 19. Conversely, if the capillary bore temperature is greater than the target temperature, computer 17 lowers the temperature of filament 167, such by decreasing the amount of current. The difference between the temperature received from sensor 68, which measures the gas temperature, and the temperature from sensor 168 is used to determine relative change in filament temperature that is required to reach the target temperature. For example, if the temperatures of the gas and capillary bores are each significantly less than the target temperature, a greater increase in the filament temperature is required than if only the capillary bore temperature is significantly less than the target temperature.

As an alternative to controlling the gas temperature by varying a filament temperature, the gas temperature can be varied by mixing a first hot gas and a second, cooler gas. By varying the ratio of the gas volumes in the mixed stream, the temperature can be varied. A mass flow controller, such as the Type 1179A General Purpose Mass Flow Controller provided by MKS Instruments of Andover, Mass., can be used to obtain and measure a variable degree of mixing between the two gas sources.

Controlling the temperature of the sample components within the capillary by use of a gas rather than by using a liquid, allows the temperature of the capillary bore (and sample components therein) to be changed much more rapidly because the temperature of the gas can be changed much more rapidly than the temperature of a liquid. It should be understood, however, that, where rapid temperature changes are not required, a liquid may be used to control the temperature of the temperature control zone.

A portion T_{cool} 66 of capillaries 33 and 19 can be provided to reduce the temperature of sample components, such as polynucleotides, after the samples have passed through the temperature control zone. Cooling the sample components can provide an increase in detection efficiency, as discussed below. The temperature in portion T_{cool} 66 can be controlled using chilled gas with an arrangement similar to that provided in the temperature control zone. Because the radial dimensions of capillaries 33, 19 are preferably on the order of about 500 microns or less, cooling the capillaries themselves serves to cool sample components migrating within the separation filling the internal bores of the capillaries. Thus, the chilled gas in the portion T_{cool} is in thermal contact with sample components present within the internal bores of capillaries 33, 19.

A fan 170 or other air circulation device is provided to introduce chilled gas into an inlet port 171. Upon entering the inlet port 171, the chilled gas comes into thermal contact with the portions capillaries 33, 19 disposed in T_{cool} and sample components present in the cooled capillary portions. The chilled gas entering input port 171 can be provided by, for example, contacting the gas with a condenser or heat exchanger filled with a chilled liquid. An outlet port 172 allows chilled gas to escape.

A sensor 173 monitors the gas temperature within T_{cool} and a sensor 174, which is in thermal contact with capillaries 33, 19, determines the temperature within the bores of the capillaries. Computer 17 preferably receives signals from sensors 173, 174. As the temperature within the temperature controlled portion of the system increases, additional cooling may be required to maintain a predetermined target temperature within T_{cool}. If computer 17 determines that the temperature within T_{cool} is greater than the target temperature, the gas flow rate through T_{cool} can be increased, such as be increasing the fan speed.

Device 40 also includes a light source 23, such as a laser emitting a wavelength suitable to generate fluorescence from the intercalating dye. A detector 25 is arranged to detect the fluorescence and provide a detector signal representative of the fluorescence intensity. The detector signal detected at a particular migration time or migration distance may be referred to as a detector coordinate. The detected fluorescence intensities are sent to the computing device 17.

Referring to FIG. 1b, a mutation detection system 500 having a thermoelectric cooler, such as a Peltier cooler 502, to cool samples that have been subjected to temperature gradient electrophoresis is shown. An example of a suitable Peltier cooler is the Thermo-Electric Module No. 01/128/040 available from Ferrotec America Corporation, Nashua, N.H. Peltier cooler 502 cools at least a portion of capillaries 33, 19 disposed in a cooled portion T_{cool} 566. A chilled side 505 of Peltier cooler 502, which is controlled by computer 517, is disposed in thermal contact with a portion of capillaries in T_{cool}. By cooling a portion of capillaries 33, 19, cooler 502 also cools samples within the cooled portions of the capillaries. As understood in the art, Peltier coolers release heat on a side that opposes the chilled side. Thus, device 500 preferably includes an apparatus, such as for circulating water or flowing gas, to remove heat from the Peltier cooler.

Thermal contact between Peltier cooler 502 and capillaries 33, 19 is preferably enhanced by using a thermally conductive material, such as a thermal paste 504, which surrounds a portion of the capillaries in contact with Peltier cooler 502. Computer 17 receives signals from a temperature sensor 503 indicative of the temperature within the internal bore of capillaries 33, 19. Computer 17 can vary the cooling level of Peltier cooler 502 by varying the current supplied to the device, as understood in the art. During operation, computer 17 compares the temperature determined by sensor 503 with a predetermined target temperature and increases or decreases the cooling level of Peltier cooler 502 if the temperature is too high or low, respectively.

Referring to FIG. 1c, a device 600 having a liquid chiller 602, is shown. An example of a suitable chiller is the MLA270 Series chiller available from Melcor, Trenton, N.J. Liquid chiller 602, which is under the control of a computer 617, circulates a chilled liquid, such as water, a poly-alcohol, or mixture thereof, through tubing 605, which is in thermal contact with the portions of capillaries 33, 19 disposed in portion T_{cool} 666. Computer 617 receives signals from a temperature sensor 603 disposed in thermal contact with capillaries 33, 19 in portion T_{cool}. When the temperature indicated by sensor 603 deviates from a predetermined target temperature, computer 617 instructs chiller 602 to decrease or increase the temperature of the liquid flowing through tubing 605 depending upon whether the sensor 603 temperature is too high or low, respectively.

The temperature and length of portions T_{cool} 66, 566, 666, hereinafter referred to collectively as T_{cool}, are preferably low enough and long enough, respectively, to allow DNA fragments that are thermally partially denatured within temperature control zone 50 to anneal prior to being detected at a reference detection zone 70 or a sample detection zone 70′. Because the system preferably uses an intercalating dye that is selective for double stranded DNA fragments, allowing denatured fragments to substantially re-anneal enhances the detection sensitivity. The temperature of T_{cool} is reduced to less than about 35° C., preferably less than about 25° C., more preferably less than about 20° C., and most preferably less than about 15°.

In any embodiment of the present invention, electrophoresis data of samples is preferably obtained simultaneously with the electrophoresis data of one or more references. By “simultaneously,” it is meant that the samples and reference are elecrophoresced in a total time that is at least about 25% less, preferably about 50% less, than twice the time required to sequentially electrophoresce the samples. Preferably, the sample is subjected to capillary electrophoresis in the sample capillary and a reference is subjected at substantially the same time to capillary electrophoresis in a second, different capillary.

It should be understood that the reference does not have to be electrophoresed simultaneously with the sample. Indeed, electrophoresis data of the sample can be processed with respect to previously acquired reference electrophoresis data, such as data present in a memory, look-up table, or database. For example, the previously acquired reference electrophoresis data may comprise data derived from one or more references that were subjected to temperature gradient electrophoresis either before or after the sample was subjected to temperature gradient electrophoresis.

Sample components, such as first and second pairs of polynucleotides, can be subjected to temperature gradient electrophoresis in the presence of more than one DNA staining dye. The different intercalating dyes preferably fluoresce at wavelengths that are sufficiently different to allow the presence of one of the dyes to be detected even when the other dye is also present. To simultaneously detect fluorescence from each of two or more dyes, the mutation detection detector preferably comprises a light dispersing element, such as a grating or prism, and a two-dimensional detector, such as a charge coupled device. An example of a suitable detector is described in U.S. Pat. No. 6,118,127, which is incorporated herein to the extent necessary to understand the present invention.

Each pair of polynucleotides that are separated in the presence of the two intercalating dyes comprises two member polynucleotides. Each member polynucleotide is preferably a double stranded polynucleotide, such as a heteroduplex or homoduplex DNA strand. Preferably, one of the intercalating dyes interacts preferentially with the first pair of polynucleotides and the second intercalating dye interacts preferentially with the second pair of polynucleotides. Thus, it is possible to determine the presence of both members of each of the first and second pairs of polynucleotides even if the pairs do not become spatially resolved during electrophoresis.

Separation Media

A preferred separation medium for mutation detection comprises a buffer, such as 1×TBE buffer, which can be prepared, for example, by dissolving 8.5 g premixed TBE buffer powder (Amerosco, Solon, Ohio) into 500 ml dionized water. An intercalating dye, such as Ethidium bromide is incorporated into the TBE buffer at a concentration sufficient to provide detection of double stranded DNA in the sample. The suitable dye concentration depends upon the particular sample and can be determined by, for example, varying the dye concentration in a series of standard samples to obtain a calibration curve of intensity versus dye concentration. As an alternative to an intercalating dye, a dye that covalently binds to the DNA can be used. An intercalating dye is preferred, however, at least because the intercalating dye can be added to the running buffer. Thus, a separate step to tag the strands of DNA is not required.

The present invention preferably allows mutation detection of DNA fragments from PCR products without first desalting or substantially purifying the products, such as by a filtration or pre-separation. In particular, the present method can be performed without removing single stranded DNA from the PCR products. This is especially important in mutation detection because the samples usually contain other biological tissues, cells, or reagents. Thus, memory effects and impurities are more of a concern in mutation detection as opposed to DNA sequencing. Sampling PCR reaction products, which may contain single strand sequences of DNA, without first desalting or purifying the products is made possible at least in part by the use of an intercalating dye, which preferably associates selectively with double stranded DNA rather than single stranded DNA. The PCR products would have to be depleted of single stranded DNA if traditional dye labels were used because the fluorescence signals from the labeled single strands would interfere with detection of the desired double stranded fragments.

Additionally, the present mutation detection device is preferably configured to inject a high pressure fluid through each separation capillary to reduce memory effects from previous analyses.

A sieving matrix can be prepared using Polyvinylpyrrolidone (PVP) which is available from Sigma (St. Louis, Mo.). A preferred sieving matrix can be made by dissolving about 0.5% to about 6% (w/v) of 360,000 M PVP into 1×TBE buffer with the intercalating dye. Preferably, the amount of PVP is about 3% (w/v). The viscosity of a three percent solution is less than 10 cp. The use of polyvinylpyrrolidone makes the capillary regeneration process very easy to implement. The capillaries have a negligible failure rate even over several months. The excellent EOF suppressing effect of the PVP medium enhances the reproducibility of decreases uncertainty associated with mutation detection. Alternatively the separation medium includes other sieving matrices such as polyacrylamide gels.

Sample Preparation and Temperature Profile Generation

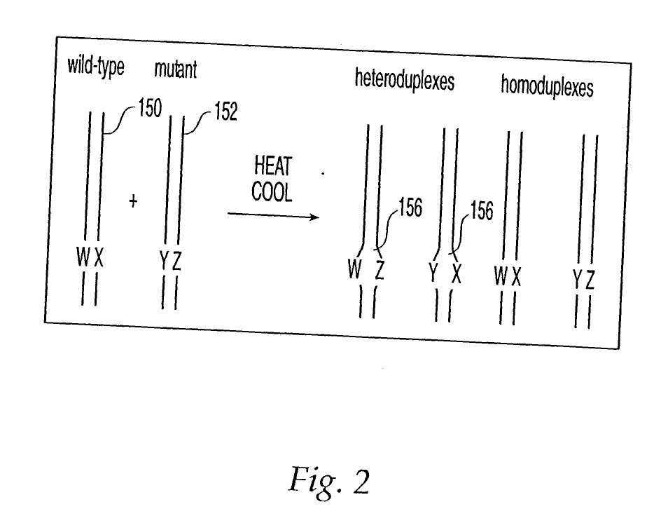

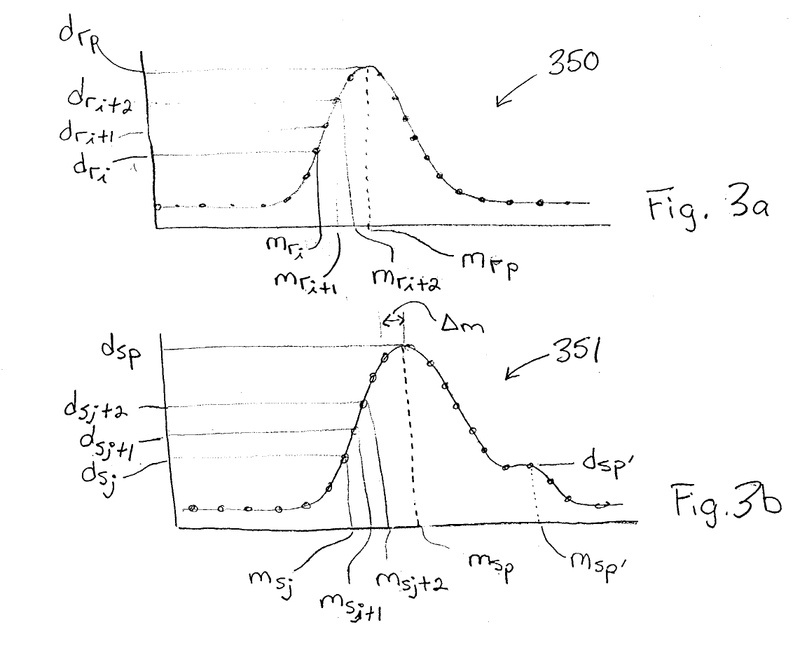

FIG. 2 illustrates the creation of a mixture of heteroduplexes and homoduplexes by hybridization. Individuals who are heterozygous in a mutation or polymorphism may have a 1:1 ratio of wild-type 150 and mutant DNA 152. The letters w, x, y, and z indicate arbitrary bases in the DNA strands. As an initial step in determining the presence of a mutation, a sample polynucleotide to be analyzed for the presence of an mutation is preferably mixed with wild-type DNA and hybridized. A mixture of heteroduplexes and homoduplexes is formed when DNA derived from an individual having two mutant alleles (homozygous mutation) is amplified.

However, DNA from homozygous wild-type individuals will form only one species, the homoduplex wild type. Thus, the presence of mutations in an individual's DNA can be detected by determining whether PCR products derived from the individual's DNA comprise heteroduplexes. Using a temperature profile of the present invention, the presence of heteroduplexes can be determined.

For sample polynucleotides containing both a heteroduplex and the corresponding homoduplex, the heteroduplex will melt (denature) at a lower temperature because the heteroduplex contains a base-pair mismatch. Melting occurs because the thermal energy of the separation medium is sufficient to overcome at least some interaction forces between a pair of DNA strands, at least partially denaturing the DNA. When the DNA becomes partially denatured, the mobility of the partially denatured strands decreases in comparison to a pair of equal length strands that are not denatured to the same extent. Therefore, the heteroduplex can be differentiated from the homoduplex by subjecting a sample to separation at a temperature sufficient to melt the heteroduplex but not the homoduplex.

During a separation performed with a ramped temperature profile, the temperature of the separation medium is increased from an initial value that is less than the melting temperature of both the homoduplex and the heteroduplex. As the temperature is raised, the heteroduplex exhibits a retarded migration behavior near its melting temperature compared to the homoduplex. Thus, the two species begin to separate. As the temperature is raised above the melting temperature of the homoduplex, the homoduplex also denatures and the difference in mobilities between the pair of compounds is reduced. Thus, the extent of separation between a homoduplex and heteroduplex depends in part on the total amount of time the separation medium is at a temperature above the melting point of the heteroduplex but less than the melting temperature of the homoduplex. The mutation can be identified by the difference in the resulting electrophoretic patterns between the homoduplex and the heteroduplex.

A temperature profile of the invention preferably includes at least one change in the temperature of the separation medium as a function of time. Temperatures during the temperature profile can be varied over any time and temperature range sufficient to induce a mobility differential between samples to be separated. In some cases, the analysis objective is to determine if any mutations are present in a sample and the melting temperatures of any heteroduplex-homoduplex pairs that would indicate presence of a mutation are not known before the analysis. Here, the temperature is preferably ramped over a wide range that encompasses the melting temperatures of substantially all heteroduplex-homoduplex pairs that might be present in the sample. In other cases, the analysis objective is to determine whether a sample contains a mutation of a particular type. In this situation, the melting temperatures of a heteroduplex-homoduplex pair that would be indicative of the mutation, if present, are known. As discussed below, the slope of the temperature profile can be optimized to enhance detection of predetermined mutation.

During electrophoresis, the temperature is preferably above the freezing point of the separation medium, such as above about 0° C., and below the boiling point of the separation medium, such as below about 100° C. The temperature within the temperature control zone is preferably substantially constant along a dimension of the separation medium that is perpendicular to the direction of migration. Thus, for example, the temperature is substantially constant across the radial dimensions of a capillary. By substantially constant temperature it is meant that the spatial temperature variations are insufficient to introduce measurable mobility variations for compounds disposed at different spatial locations within the temperature control zone at any given instant. Thus, at any given instant, the temperature at any point along the portion of each capillary within the temperature control zone is preferably constant, i.e., there are substantially no spatial temperature gradients in the temperature control zone.

For accurate comparison of the patterns, a reproducible temperature profile is required. Because in this invention the temperature of the separation medium can be varied independently of the electric field, arbitrary temperature profiles can be selected without negatively perturbing mutation detection performance. For example, for the separation of heteroduplex sample compounds using an apparatus and temperature profile of the present invention, migration times have a relative standard deviation of less than 2%.

Because the mobility retardation (differential mobilities between a heteroduplex and corresponding homoduplex) occurs only when the DNA fragments begin to melt, the part of the capillary that is not elevated above the melting temperature of a fragment, will not affect the differential mobility of the fragments. Preferably, a temperature profile of the invention is not begun until at least some and preferably substantially all fragments in a sample have migrated into the temperature control zone.

In order to generate a reasonably accurate range over which to vary the temperature and the rate of temperature variation, the configuration of the capillary layout has to be considered. Preferably, the temperature range and variation rate are appropriate to allow determination of substantially any mutation in any of the unknown samples being analyzed.

Parameters for a temperature ramping profile preferably include the (1) temperature ramping range from a low temperature T_{L} to a higher temperature T_{H}; (2) time, tᵣ, after injection at which the temperature ramp is initiated; and (3) rate, r, at which the temperature is ramped.

Preferred procedures for determining temperature ramping parameters include (1) selection of the separation voltage and (2) selection of a sample standard that includes DNA fragments covering the size range of fragments in the samples to be analyzed. The voltage depends on the sieving matrix used, the sizes of the fragments to be separated, and the length of the separation lane, as understood in the art.

It should be emphasized that temperature profiles suitable for use with the mutation detection device do not have to be a linear function of time but may also be non-linear or include a combination of profile segments that each have a same or different temperature gradient and duration.

Detection of Mutations

One embodiment of the present invention relates to an electrophoresis system that includes a computer or other processor configured to determine the presence of an SNP or mutation in a biological sample. In another embodiment, the processor is independent of an electrophoresis system. In any event, the processor is typically implemented through a combination of hardware and executable software code. In the usual case, the processor includes a programmable computer, perhaps implemented as a reduced instruction set (RISC) computer, which handles only a handful of specific tasks. The computer is typically provided with at least one computer-readable medium, such as a PROM, flash, or other non-volatile memory to store firmware and executable software code, and will usually also have an associated RAM or other volatile memory to provide work space for data and additional software.

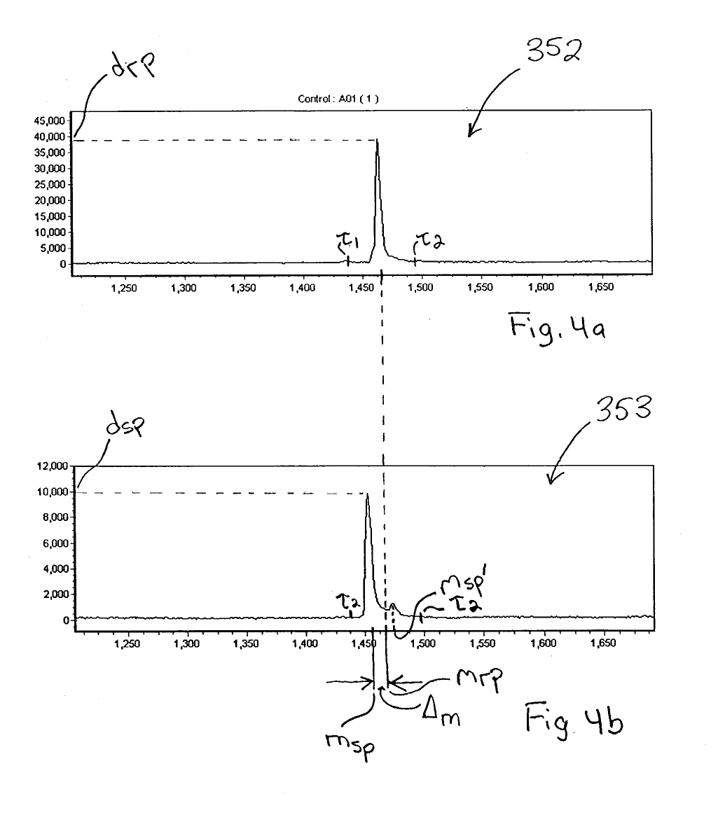

Referring to FIGS. 3a and 3b, a set of electrophoresis data preferably includes a plurality of points each having a detector coordinate and, optionally, a migration coordinate. For example, reference electrophoresis data 350 of FIG. 3a include a plurality of points each having a detector coordinate dri and a migration coordinate mri. Thus, the ith detector coordinate corresponds to the ith migration coordinate. Similarly, sample electrophoresis data 351 of FIG. 3b include a plurality of points each having a detector coordinate dsi and a migration coordinate msi.

For both reference and sample electrophoresis data, the detector coordinate results from a detector signal, such as a fluorescence intensity, and is indicative of whether or not a compound, such as a sample or reference, is present in the detection zone. The detector coordinate may also be indicative of the amount of a component present in the detection zone.

The migration coordinate is indicative of a location of a given electrophoresis data point along a separation dimension. Exemplary migration coordinates include migration time, migration distance, and mobility. It should be understood, however, that the migration coordinate of a given detector coordinate may be determined from the position of the detector coordinate relative to other detector coordinates of the detector signal or electrophoresis data. Thus, a set of electrophoresis data need not include a separate plurality of migration coordinates. A detector signal may be equivalent to a set of electrophoresis data. For example, a detector signal or set of electrophoresis data may each include one or more vectors or equivalent thereto each comprising a plurality of detector coordinates.

As used herein, the term sample indicates a sample that is to be analyzed to determine or confirm the presence of a mutation in one or more components of the sample. Preferred samples preferably includes one or more sample polynucleotides, i.e., polynucleotides to be analyzed to determine or confirm the presence of mutation therein. Preferred references include one or more polynucleotides have a known mutation status. The reference may comprise, for example, a molecular ladder, mutation standards comprising a particular set of fragments, or a combination thereof. Upon comparing the electrophoresis data derived from the sample with those of the reference, it is possible to determine or confirm the presence of mutation in the sample.

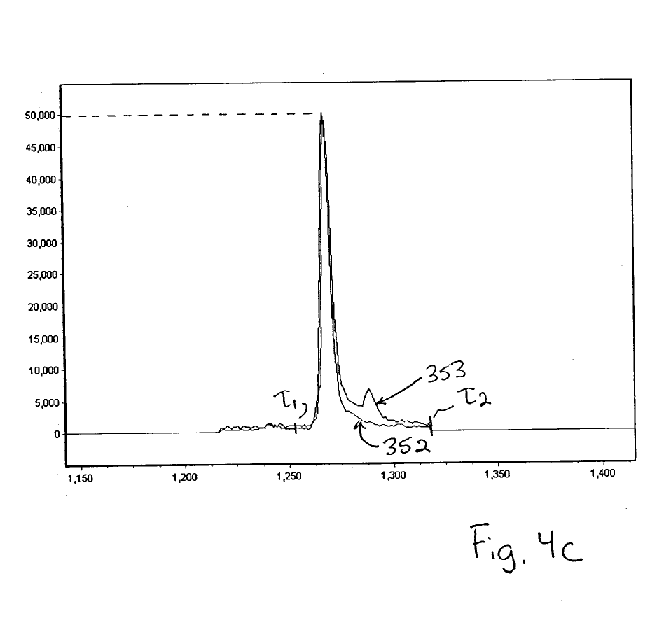

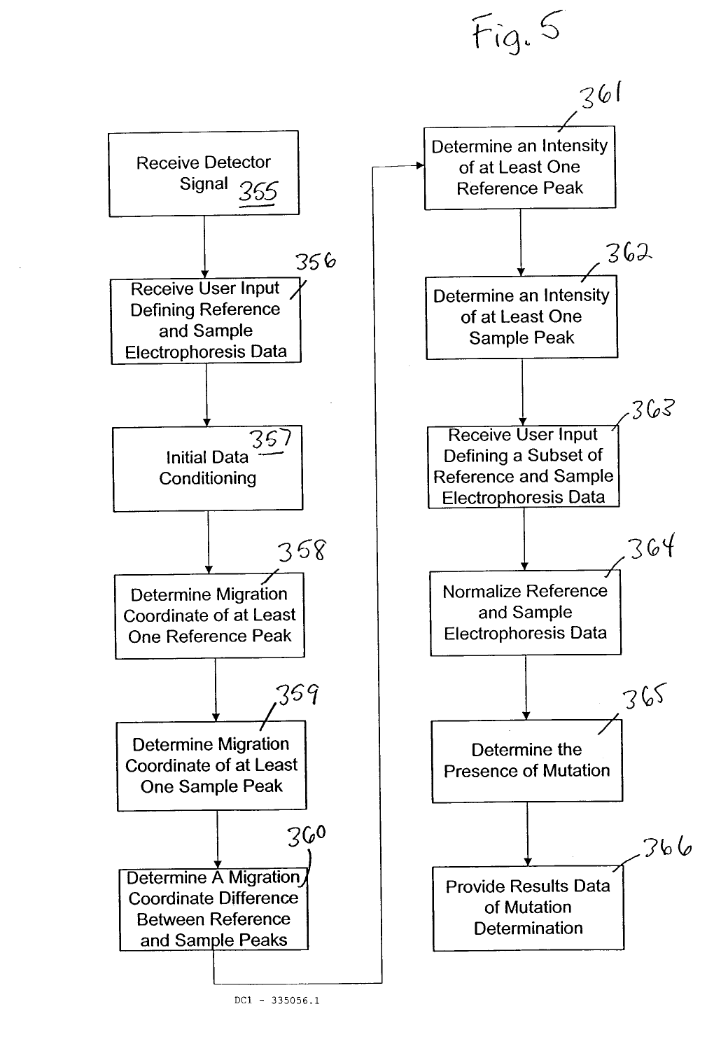

Various steps that may be carried out by the computer or other processor in response to code of a computer-readable medium are discussed below. Although electrophoresis data are typically comprised of a plurality of individual points, electrophoresis data, as seen in FIGS. 4a-4c are typically presented in a form that at least partially obscures the presence of individual points in the data.

It should be understood that various steps may be combined and/or executed in orders other than discussed herein. Moreover, a method or code in accordance with the present invention may include or execute only a subset of the steps discussed herein. Of course, a method or code in accordance with the present invention may include or execute steps additional to those discussed herein.

Receive Detector Signal

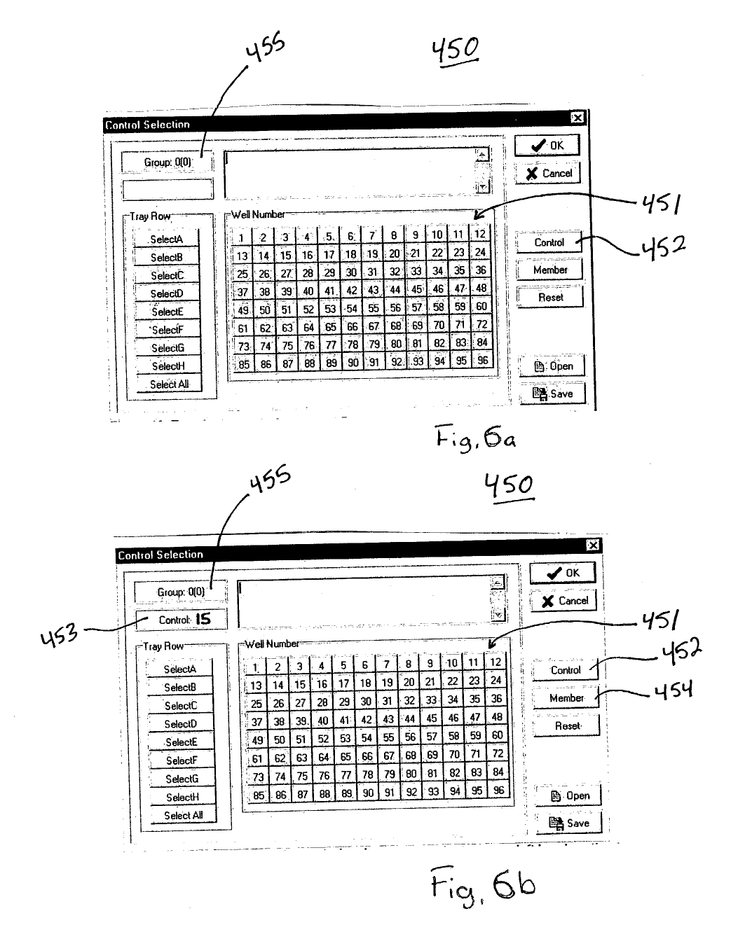

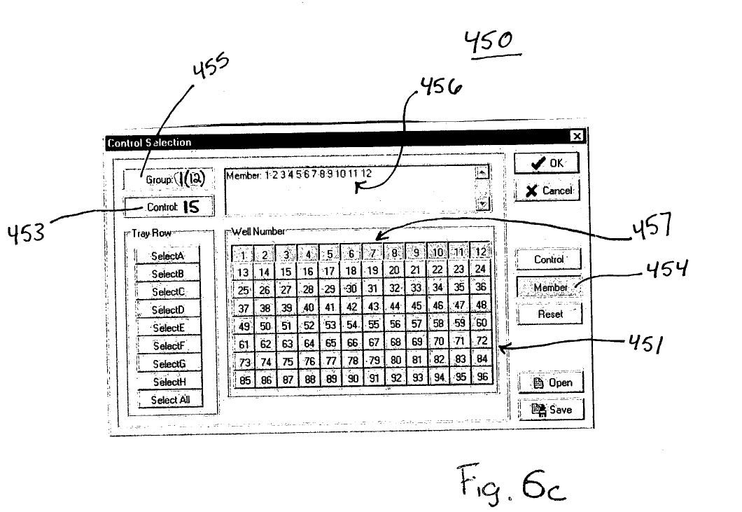

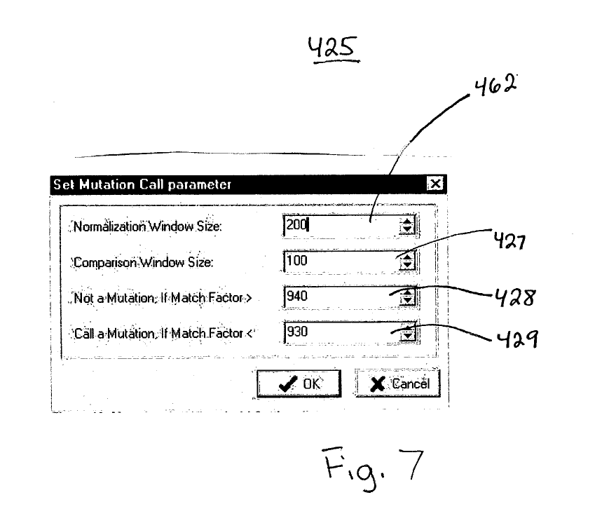

Referring to the flow chart of FIG. 6, the computer or processor may be configured to receive 355 detector signals obtained from TGE of at least one reference compound and TGE of at least one sample compound. Because the computer may be either local to the electrophoresis instrument or remote therefrom, the detector signals may be provided in using, for example, a hardwired connection, wireless connection, a network, a storage medium such as a disk or memory, or combination thereof.

The code of the computer readable medium may include code configured to convert detector signals to reference and sample electrophoresis data. Because the detector may output detector signals already in the form of electrophoresis data a conversion step may not be necessary so that the computer or processor may merely receive the electrophoresis data. Detector signals in which the relative positions of detector coordinates thereof may be determined are considered equivalent to electrophoresis data.

Receive Input Defining of Reference and Sample Electrophoresis Data

Because the present invention contemplates processing, possibly essentially simultaneously, a plurality of sets of sample electrophoresis data and reference electrophoresis data, the code is preferably configured to receive 356 user input defining one or more groups of sample electrophoresis data. The received input may also define one or more groups of reference electrophoresis data to be used in processing respective groups of sample electrophoresis data. For example, the electrophoresis data to be processed may include a number N sets of data. A number Nr of the N sets of data, where 1≦Nr≦N, are reference electrophoresis data. A number Ns of the N sets of data, where 1≦Ns≦N−Nr, are to sample electrophoresis data. The code preferably allows the user to define a number Nsg groups of sample electrophoresis data, where 1≦Nsg≦Ns. The code preferably allows the user to define at least one set of reference electrophoresis data for processing each of respective groups of sample electrophoresis data. Thus, the number Nr of sets of reference electrophoresis data is preferably at least as large as the number Nsg of groups.

FIGS. 6a-6c show an exemplary display 450 by which a user may define groups of sample electrophoresis data obtained from an electrophoresis array device having an array of 96 separation lanes. A well number table 451 includes 8 rows of 12 indicia each corresponding to a particular well of a sample tray used to introduce samples, references, and other materials to separation lanes of the array. The well number corresponds to the separation lane number of the array and, therefore, to the type of electrophoresis data (whether sample or reference) obtained from a particular separation lane.

The code is configured to define either automatically or in response to input received from a user one or more groups of sample electrophoresis data each having one or more member sets of sample electrophoresis data. The processor may automatically execute code displaying a prompt for user input or the user input may be received subsequent to the user's manipulation of a displayed control 454. Thus the input defining the members of a group may be received upon, for example, the user's manipulation of one or more of the indicia of well number table 451. As seen in FIG. 6c, for example, control 454 and a row 457 have been manipulated thereby defining member sets of a group of sets of sample electrophoresis data. A group indicium 455 indicates information related to the defined group, such as a group designation and the number of members of the group. Identities, such as well or separation lane numbers, of members of the group may be displayed in a table 456.

The code is configured to receive user input indicative of reference electrophoresis data to be used to process members of a defined group of sample electrophoresis data. The user input may be received subsequent to the user's manipulation of a displayed control 451. Thus, the input may be received upon a user's manipulation of one of the indicia of well number table 451. As seen in FIG. 6b, a control indicium 453 indicates that a user has selected electrophoresis data obtained from the 15^{th} separation lane to be used as the reference electrophoresis data for processing a group of separation electrophoresis data.

Initial Conditioning

In certain situations, detector signals or electrophoresis data therefrom must be subjected to initial conditioning 358, such as by data smoothing, baseline subtraction, or by using deconvolution techniques to identify overlapped peaks. This may be the case with either or both of the reference and sample electrophoresis data. Suitable data conditioning techniques, such as those discussed below, are disclosed in U.S. application Ser. No. 09/676,526, filed Oct. 2, 2000, titled Electrophoretic Analysis System Having in-situ Calibration, which application is hereby incorporated to the extent necessary to understand the present invention. The computer-readable medium includes code to perform such conditioning.

Smoothing can be accomplished by using, for example, a Savitzky-Golay convoluting filter to improve the signal to noise ratio. Optimal properties of the filter, such as the width and order, can be determined by a user of the present invention on the basis of the signal to noise ratio of the data and the widths of peaks in the data.

Baseline subtraction can be performed to eliminate baseline drift. Typically, minima are identified in successive local sections of data, e.g., every 300 data points. Two or more minima in adjacent sections are connected, such as by a straight line or a polynomial fit to the minima. The values along the line connecting the minima are then subtracted from the intervening raw data. The new values after the baseline subtraction and smoothing are stored for further processing. The order of data smoothing and baseline subtraction can be reversed.

Overlapped peaks within the separations data can be identified and resolved using peak-fitting techniques. In most electrophoresis separations, the earlier-detected peaks are narrower than the later-detected, slower moving peaks. Within a given local section of data, however, peaks due to the presence of a single fragment have similar widths. Moreover, adjacent peaks rarely overlap exactly. Rather, the overlapped peaks a generally offset from one another. Accordingly, peaks due to the presence of multiple fragments tend to be wider than the single fragment peaks. Once a region of data containing overlapped peaks is identified, the underlying peaks can be resolved by fitting a model of the data to the observed data. Typically, the peak fitting model includes parameters that describe the amplitude, position, and width of each underlying peak.

Determine Migration Coordinate of at Least One Reference Peak

The code may be configured to locate 358, either automatically or in response to user input, at least one reference peak in the reference electrophoresis data. Reference peaks correspond to the presence of at least one reference compound that has been subjected to TGE. By locate, it is meant determine the position of the reference peak amongst the reference electrophoresis data. For example the code may be configured to fit the reference peak to a peak shape model and determine the migration coordinate from the fitted parameters. Generally, the fitting parameters include the migration coordinate of the peak, the peak maximum, and peak width. For example, referring to FIG. 3a, the migration coordinate mᵣₚ of the reference peak does not exactly coincide with any of the migration coordinates mᵣᵢ of the reference electrophoresis data but may determined from parameters of a peak shape function, such as a Gaussian function.

Locating the reference peak may include receiving a user input indicative of the migration coordinate of the reference peak. Locating the reference peak may also include finding the reference peak, such as by seeking detector coordinates, such as intensity values, for various migration coordinates that exceed a threshold indicative of the presence of a peak.

Determine Migration Coordinate of at Least One Sample Peak

The code may be configured to locate 359, either automatically or in response to user input, at least one sample peak in each set of sample electrophoresis data. Sample peaks correspond to the presence of at least one sample compound that has been subjected to TGE, as seen in FIG. 4b. By locate, it is meant determine the relative position of the sample peaks amongst the sample electrophoresis data, for example by determining a migration coordinate mₛₚ, such as a migration time or distance, of the sample peak. The at least one sample peak may be located using identical or similar code and methods used to locate the at least one reference peak.

Determine A Migration Coordinate Difference Between the Reference and Sample Peaks

Referring to FIGS. 4a and 4b, the reference and sample peaks may be located at migration coordinates that differ by an amount Δₘ. For example, the reference peak may have a migration time of τ_{τ} while the sample peak may have a migration time τₛ=τᵣ±Δ_{τ}. The code may be configured to determine Δ_{τ} in a step 360. The migration coordinate difference may also be expressed in a number of electrophoresis data points separating the reference and sample peaks.

The code may be further configured to align the reference and sample electrophoresis data based on the migration coordinate difference Δₘ. For example, where the sample peak has a migration coordinate τₛ=τᵣ+Δ_{τ}, the code may be configured to subtract an amount Δ_{τ} from each migration coordinate of the sample electrophoresis data.

Aligned, normalized, and overlaid sample and reference electrophoresis data may be displayed as seen in FIG. 4c.

Determine an Intensity of the at Least One Reference Peak

The code may be configured to determine 361 a detector coordinate indicative of an intensity of the reference electrophoresis data. The detector coordinate is preferably indicative of an intensity, such as the maximum intensity dᵣₚ, of the at least one reference peak of the reference electrophoresis data. For example, the detector coordinate may be determined by locating the reference data point that has the maximum detector coordinate value among the reference data points defining the reference peak. Alternatively, the detector coordinate indicative of an intensity may be determined from one or more fitted parameters obtained by fitting the reference peak to a peak shape function.

Determine an Intensity of the at Least One Sample Peak

The code may be configured to determine 362 a detector coordinate indicative of an intensity of the sample electrophoresis data. The detector coordinate is preferably indicative of an intensity, such as the maximum intensity dₛₚ, of the at least one sample peak of the sample electrophoresis data. The detector coordinate indicative of an intensity of the sample electrophoresis data be determined using identical or similar code and methods used to determine the detector coordinate indicative of the intensity of the reference electrophoresis data.

Determine a Size of a Subset of Electrophoresis Data for Processing

The code may be configured to determine 363 a size of a subset of sample electrophoresis data and reference electrophoresis data to be processed for determining the presence of mutation in one or more sample polynucleotides. In determining the presence of mutation, the code preferably processes only sample and reference electrophoresis data within the subset of data. Use of a subset of the electrophoresis data enhances the precision of mutation determination because electrophoresis data not relevant to differences between a reference peak and a sample peak is excluded from the process of determining whether the sample polynucleotide includes a mutation.

The size of the subset of data is preferably defined relative to migration coordinates of the electrophoresis data. For example, the subset of data may be defined with respect to migration time, distance or simply a number of data points. The subset of data is preferably centered about the same position within the electrophoresis data as the normalization window discussed elsewhere herein. For example, the subset of data may be centered about a peak present in the data.

As discussed below, each set of sample electrophoresis data and reference electrophoresis data may respectively include more than one sample and reference polynucleotide. Thus, the code is preferably configured to select, either automatically or using a user input, a plurality of subsets of each set of sample and reference electrophoresis data. For example, where the reference and sample electrophoresis data each include peaks of two different polynucleotides, the code may determine the locations of the subsets of data based upon the locations of a plurality of peaks. The size of the subsets of data may be determined also be determined automatically or from user input.

Automatic determination of subset size may be based on a width of peaks present in either or both sets of reference and sample electrophoresis data. For example, the code may determine a peak width, such as a full width at half maximum of a peak present in the reference electrophoresis data, and determine the size of the subset of data for processing based on the peak width. The code may be configured to receive user input indicative of the relationship between the size of the subset of data relative to the peak width.

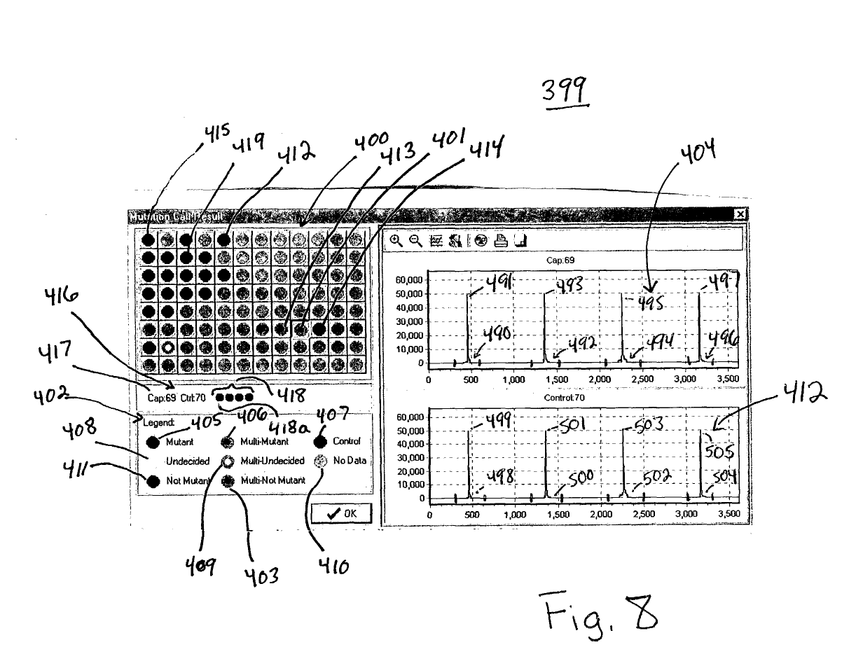

Referring to FIG. 7, the code may be configured to receive user input indicative of the size of a subset of data. For example, a display window 425 includes a control 426 by which a user may enter a size of a subset of data for processing.

Normalize the Reference and Sample Electrophoresis Data

The code may be configured to normalize 364 the reference and sample electrophoresis data based on at least one detector coordinate of the reference electrophoresis data and at least one detector coordinate of the sample electrophoresis data. The detector coordinates used to normalize the electrophoresis data are preferably indicative of respective intensities, such as fluorescence intensities, of the reference and sample electrophoresis data. Most preferably, the detector coordinates used to normalize the electrophoresis data are indicative of respective peak intensities, such as the maximum intensities, of the reference and sample peaks.

The reference and sample electrophoresis data may be normalized with respect to one another, such as by dividing detector coordinates one of the reference and sample electrophoresis data by a detector coordinate of the other data. Alternatively, the reference and sample electrophoresis data may each be normalized with respect to a predetermined value, as seen in FIG. 4c.

The code may be configured to receive user input indicative of the size of a subset of data to be normalized, i.e., a normalization window. For example, a display window 425 includes a control 426 by which a user may enter a size of a subset of data for processing.

Determine the Presence of Mutation

The code is preferably configured to process reference electrophoresis data and sample electrophoresis data to determine 365 the presence of mutation in one or more sample polynucleotides underlying sample peaks in the sample electrophoresis data. As discussed more fully below, results data obtained from the processing may include one or more values indicative of the presence of mutation. Referring to window 425 of FIG. 7, the code may be configured to receive user input indicative of a threshold that may be compared with a value determined from processing the electrophoresis data for determining whether a mutation is or is not present in a sample polynucleotide. For example, a control 428 allows the processor to receive a user entered threshold for determining the absence of a mutation. A control 429 allows the processor to receive a user entered threshold for determining the presence of a mutation. Where two different thresholds are selected, as shown in FIG. 7, a value obtained from a mutation determination may fall intermediate between the thresholds. In such as case, the code may be configured to indicate that the mutation determination was indeterminate or undecided, as discussed below. Alternatively or in combination with such indication, the code may perform further processing of the data and/or provide a user with the option to determine the presence of mutation by eye.

Referring to FIGS. 4a and 4b, sample electrophoresis data 353 include multiple peaks 354 that do not all appear in the electrophoresis data of the reference electrophoresis data 352. At least one extra peak appears within a migration time τ₁ and a migration time τ₂. The time between migration time τ₁ and migration time τ₂ is an example of a subset of the sample and reference electrophoresis data that may be processed to determine the presence of mutations in sample polynucleotides of the sample electrophoresis data.

Even a slight change in the pattern of peaks is sufficient to be indicative and determinate a mutation in the sample because TGE performed using the mutation detection apparatus of the present invention is highly reproducible. Perfect separation of the fragments in the heteroduplex samples, however, is not necessary to identify the presence of a mutation. For example, the presence of a mutation may be indicated because a peak of the sample electrophoresis data has a width broader that a width of a peak of reference electrophoresis data, where the reference is free of a mutation. Code in accordance with the presence invention may be configured to determine a width of respective sample and reference peaks to determine the presence of mutation in a sample. Peak widths may be determined, for example, a full width half maximum, as understood in the art.

The code may be configured to determine a similarity between the reference and sample electrophoresis data. The code may be configured to determine the presence of a single polynucleotide polymorphism or mutation in the sample based on the similarity. Where the reference electrophoresis data results from TGE of a reference polynucleotide not having a mutation, greater similarity between the reference electrophoresis data and sample electrophoresis data is indicative of the absence of mutation in the sample polynucleotide. Greater dissimilarity is indicative of the presence of mutation in the sample polynucleotide. Of course, a polynucleotide having a mutation may be used to obtain the reference electrophoresis data.

In one embodiment, determining similarity comprises determining at least one difference between (1) a product of at least one detector coordinate of the reference electrophoresis data and at least one detector coordinate of the sample electrophoresis data and (2) a product from a value determined from a plurality of the detector coordinates of the reference electrophoresis data and a value determined from a plurality of detector coordinates of the sample electrophoresis data. The difference is preferably squared. For example, a similarity may be determined as a covariance cov(dᵣ dₛ) between the reference and electrophoresis data:

$$cov ( {d}_{r} {d}_{s} ) = k \times {[ {\sum }_{ij} {d}_{{r}_{i}} \times {d}_{{s}_{j}} - \frac{{\sum }_{i} {d}_{{r}_{i}} \times {\sum }_{j} {d}_{{s}_{j}}}{N} ]}^{2} \qquad ( 1 )$$

where the dᵣᵢ are detector coordinates of the reference electrophoresis data each corresponding to a migration coordinate mᵣᵢ, the dₛⱼ are detector coordinates of the sample electrophoresis data each corresponding to a migration coordinate mₛⱼ, N is the number of data points of the electrophoresis data, and k is a predetermined constant, which may have on any value but is preferably an integer from 1 to 1000.

Covariance between the sample and reference electrophoresis data may also be expressed as:
cov(dᵣdₛ)=\overline{dᵣdₛ}−\overline{dᵣ}\overline{dₛ} (2)
where \overline{dᵣdₛ} is the average of the products of respective detector coordinates of the reference and sample electrophoresis data and \overline{dᵣ}\overline{dₛ} is the product of the averages of the detector coordinates of the reference and sample electrophoresis data.

The similarity may be weighted by a value indicative of the variability of one or both of the reference and sample electrophoresis data. Exemplary measures of the variability of data include the standard deviation and mean absolute deviation. In one embodiment, the similarity may be expressed as the correlation p between the reference and sample electrophoresis data:

$$ρ = \frac{cov ( {d}_{r} {d}_{s} )}{{σ}_{{d}_{r}} {σ}_{{d}_{s}}} \qquad ( 3 )$$

where σ_{dr} is the standard deviation of the detector coordinates of the reference electrophoresis data and σ_{ds} is the standard deviation of the detector coordinates of the sample electrophoresis data. For example, the correlation may be determined by:

$$ρ = \frac{k \times {[ {\sum }_{ij} {d}_{{r}_{i}} \times {d}_{{s}_{j}} - \frac{{\sum }_{i} {d}_{{r}_{i}} \times {\sum }_{j} {d}_{{s}_{j}}}{N} ]}^{2}}{{[ {\sum }_{i} {d}_{{r}_{i}}^{2} - \frac{{\sum }_{i} {d}_{{r}_{i}} \times {\sum }_{i} {d}_{{r}_{i}}}{N} ]}^{2} \times {[ {\sum }_{j} {d}_{{s}_{j}}^{2} - \frac{{\sum }_{j} {d}_{{s}_{j}} \times {\sum }_{j} {d}_{{s}_{j}}}{N} ]}^{2}} \qquad ( 4 )$$

The correlation is indicative of whether a mutation is present in the sample electrophoresis data. For example, using a value of k=1000, a value of 0 determined from Equation 4 indicates that there is no match between the electrophoresis data and a value of 1000 indicates that the electrophoresis data are identical. The value determined from Equation 4 is referred to herein as a match factor. If the reference electrophoresis data are representative of a separation of a compound not including a mutation, smaller match factors are indicative of a decreasing probability that the sample includes a mutation. The match factor is an example of a value determined from a mutation determination that may be compared to the threshold discussed above.

In another embodiment for determining a similarity between the reference and sample electrophoresis data, the code may be configured to determine a plurality of detection coordinate differences Δ_{d} between detector coordinates of the reference and sample electrophoresis data. For example, the kth detection coordinate difference Δₖ may indicative of a difference between (1) the detection coordinate dᵣᵢ of the reference electrophoresis data point having the migration coordinate mᵣᵢ and (2) the detection coordinate dᵣⱼ of the sample clectrophoresis data point having the migration coordinate mₛⱼ. The k^{th}+1 detection coordinate difference Δₖ₊₁ is indicative of a difference between (1) the detection coordinate dᵣᵢ₊₁ of the reference electrophoresis data point having the migration coordinate mᵢ₊₁ and (2) the detection coordinate dₛⱼ₊₁ of the sample electrophoresis data point having the migration coordinate mₛⱼ₊₁. The detection coordinate differences may be squared and summed. A greater sum of detection value differences is indicative of increasing dissimilarity between the reference and sample electrophoresis data. If the reference electrophoresis data are representative of a separation of a compound not including a mutation, a increased dissimilarity is indicative of an increased probability that the sample includes a mutation.

Reference electrophoresis data and sample electrophoresis data may exhibit high similarity even though one of the data correspond to a lack of mutation while the other data correspond to the presence of mutation. In these cases, a covariance or correlation that is intermediate between purely similar or entirely dissimilar data may be obtained. The code is preferably configured to determine a number of peaks present in the reference electrophoresis data and sample electrophoresis data in order to determine whether a mutation is present in the sample.

The presence of a peak in the electrophoresis data may be determined by seeking detector coordinates within the data that exceed a peak threshold. The threshold may be set manually or may be determined automatically on the basis of variability within the data. Once detector coordinates exceeding the threshold are found, the code may seek to fit a region of data in the vicinity of these detector coordinates to one or more peak shape models determine whether a peak is present. For example, referring to FIG. 4b, the sample electrophoresis data include a second smaller peak having a migration coordinate mₛₚ, and a detector coordinate dₛₚ.

Determining a number of peaks may include selecting, manually or automatically, a subset of the electrophoresis data as discussed above. For example, with respect to migration time a migration time of a detected peak, the size of the subset may be about 15%, preferably about 10% of the migration time of the peak identified in the reference electrophoresis data. The subset of data is preferably centered about the peak in the reference electrophoresis data.

The number of peaks appearing within the subset of sample electrophoresis data is determined and compared to the number of peaks in the subset of reference electrophoresis. Typically, there is only one peak in the subset of the reference electrophoresis data. If the number of peaks in the subset of the sample electrophoresis data exceeds the number of peaks in the subset of the reference electrophoresis data, the presence of mutation is indicated. Obviously, if one obtains a negative result in determining the presence of a mutation in an unknown sample, then the absence of a mutation in the unknown sample has been determined.

Provide Results Data of Mutation Determination

Referring to FIG. 8, the code may be configured to display results data indicative of the mutation status of polynucleotides subjected to TGE. As an alternative or in combination with display of the results data, the code may be configured to write the results data to a storage medium, such as a memory, storage disk, hard drive, or printer.

The results data may be displayed by code executing for a graphical user interface 399 that is preferably configured to receive user input and, more preferably, prompt a user for input relating to the display and processing of electrophoresis data obtained from TGE of sample and reference polynucleotides. Exemplary aspects of results data display are discussed below.

An exemplary status table 400 of user interface 399 includes a plurality of status indicia each indicative of the mutation status of polynucleotides subjected to TGE along separation lanes of an electrophoresis apparatus having a plurality of separation lanes. Thus, status table 400 includes 96 status indicia displayed in 8 rows of 12 status indicia each. A status indicium 401, for instance, is indicative of the mutation status of sample polynucleotides subjected to TGE along the 69^{th} separation lane.

The code may be configured to display a legend 402 having one or more legend indicia to which a user may refer to interpret status indicia of status table 400. For example, status indicium 401, which corresponds to a legend indicium 403, indicates that at least two sample polynucleotides where subjected to TGE in the 69^{th} capillary and that the at least two sample polynucleotides were determined not to include mutations. Thus, in addition to indicating the mutation status of polynucleotides of the 69^{th} separation lane, status indicium 401 preferably further indicates that more than one sample polynucleotide was subjected to TGE therealong.

A legend indicium 405 indicates that a single sample polynucleotide subjected to TGE along a corresponding separation lane includes a mutation. Status indicium 419 representative of the 5^{th} separation lane corresponds to the mutation status indicated by legend indicium 405. A legend indicium 406 indicates that, of a plurality of sample polynucleotides subjected to TGE along a corresponding separation lane, at least one includes a mutation. Status indicium 413 representative of the 68^{th} separation lane corresponds to the mutation status indicated by legend indicium 405.

Where a plurality of polynucleotides are subjected to TGE along a given separation lane, the code may display, preferably but not essentially in a second status table, status indicia indicative of the mutation status of respective polynucleotides of the separation lane. For example, a second status table 416 includes indicia 417 that are indicative of the separation lanes to which the second status table 416 presently corresponds. In FIG. 8, second status table 416 is shown as corresponding to separation lanes 69 and 70. Second status table 416 also includes status indicia 418, which are indicative of the mutation status of respective sample polynucleotides of a separation lane. In the second status table 416, respective status indicia 418 indicate that each of four sample polynucleotides of the 69^{th} separation lane are free of mutation.

A legend indicium 411 indicates that a sample polynucleotide subjected to TGE along a corresponding separation lane was free of mutation. A status indicium 415, for example, representative of the mutation status of the 1^{st} separation lane corresponds to the mutation status indicated by legend indicium 411.

Other status and legend indicia may relate to reference polynucleotides. A legend indicium 407, for example, indicates that one or more reference polynucleotides were subjected to TGE along a corresponding separation lane. A status indicium 414, for example, indicates that at least one reference polynucleotide was subjected to TGE along the 70^{th} separation lane.

A legend indicium 410 indicates the absence of electrophoresis data for a particular separation lane. Legend indicium 410 may be displayed where, for example, only a subset of available separation lanes are used for a particular analysis.

The code may also be configured to display electrophoresis data obtained from one or more of the capillaries of the separation apparatus. Thus, sample electrophoresis data 404 includes fluorescence intensity-migration time data obtained from the TGE of a plurality of sample polynucleotides of the 69^{th} capillary. Electrophoresis data 412, which include fluorescence intensity migration time data obtained from the TGE of a plurality of reference polynucleotides, are optionally displayed along with electrophoresis data 404. It is preferred that a user may select amongst the various sets of electrophoresis data to determine those which are displayed.

As seen in FIG. 8, the sample electrophoresis data 404 may include a plurality of different sample polynucleotides each having a different migration coordinate. For example, a first subset 490 of sample electrophoresis data includes a sample peak 491, a second subset 492 includes a sample peak 493, and a third subset 494 includes a sample peak 495, and a fourth subset 496 includes a sample peak 497. Each sample peak 491, 493, 495, and 497 corresponds to the presence of a different sample polynucleotide subjected to TGE for determination of the presence of mutation. Similarly, reference electrophoresis data 412 may include a plurality of different reference polynucleotides. For example, a first subset 498 of reference electrophoresis data includes a reference peak 499, a second subset 500 includes a reference peak 501, and a third subset 502 includes a reference peak 503, and a fourth subset 504 includes a reference peak 505. Each reference peak 499, 501, 503, and 505 corresponds to the presence of a different reference polynucleotide subjected to TGE for use in determining the presence of mutation in a respective one of the sample polynucleotides underlying sample peaks 491, 493, 495, and 497. For example, the presence of mutation in a sample polynucleotide underlying peak 491 was determined using subset 498 of reference data 412. The mutation status of the sample polynucleotide underlying peak 491 is indicated by status indicium 418a.

At least the status indicia of status table 400 may preferably be displayed as “clickable buttons” that a user may manipulate to select the separation lanes for which electrophoresis data will be displayed. For example, status indicium 401, which corresponds to the electrophoresis data 404 of the 69^{th} separation lane, is shown as having been manipulated, such as by “clicking” thereon.

The mutation status of one or more polynucleotides subjected to TGE along a given separation lane may be uncertain or otherwise indeterminate based on the current mutation determination settings. Thus, for example, a legend indicium 408 indicates that the mutation status of polynucleotides subjected to TGE along a corresponding separation lane is not decided or is otherwise indeterminate. A legend indicium 409 indicates that the mutation status of a plurality of polynucleotides subjected to TGE along a corresponding separation lane were not decided or are otherwise indeterminate.

The code may be configured to receive, such as in response to a code-executed prompt, user input to allow further processing of electrophoresis data in which a mutation status was not decided or was otherwise indeterminate. The code may include an eye calling provision, which, when executed, displays electrophoresis data obtained from the separation lanes having polynucleotides with an undecided status. Electrophoresis data from a separation lane having one or more reference polynucleotides may also be displayed. Displaying the electrophoresis data allows a user to visually determine whether a mutation is present, such as be determining the extent of similarity between reference and sample electrophoresis data. Alternatively, or in combination with an eye calling provision, the code may execute other processing, such as to perform a peak counting determination as discussed above. The code preferably allows a user to edit a mutation determination for a particular sample polynucleotide. For example, if a determination was not decided, the user may enter a determination arrived at through the eye calling provision.

While the above invention has been described with reference to certain preferred embodiments, it should be kept in mind that the scope of the present invention is not limited to these. Thus, one skilled in the art may find variations of these preferred embodiments which, nevertheless, fall within the spirit of the present invention, whose scope is defined by the claims set forth below.

1. A system for determining the presence of a mutation in at least one sample polynucleotide, comprising:
a processor configured to at least:
receive (1) at least a first set of sample electrophoresis data indicative of a temperature gradient electrophoresis (TGE) separation of at least one sample polynucleotide, and (2) at least a first set of reference electrophoresis data indicative of a TGE separation of at least one reference polynucleotide; and
process at least a subset of the first set of sample electrophoresis data and at least a subset of the first set of reference electrophoresis data to prepare a set of result data corresponding to the first set of sample electrophoresis data, wherein the set of result data includes data indicative of whether a sample polynucleotide of the first set of sample electrophoresis data includes a mutation.

2. The system of claim 1, wherein the system further includes a display and the processor is configured to display result data corresponding to the first set of sample electrophoresis data.

3. The system of claim 2, wherein the displayed result data includes at least one indicium of whether a sample polynucleotide of the first set of sample electrophoresis data includes a mutation.

4. The system of claim 3, wherein displayed result data corresponding to the first set of sample electrophoresis data includes an indicium of whether the first set of sample electrophoresis data includes more than one sample polynucleotide.

5. The system of claim 4, wherein displayed result data corresponding to the first set of sample electrophoresis data includes an indicium corresponding to each sample polynucleotide of the first set of sample electrophoresis data, each indicium being indicative of whether a respective one of the sample polynucleotides includes a mutation.

6. The system of claim 3, wherein the processor is configured to at least:
receive at least a second set of sample electrophoresis data;
process at least a subset of the second set of sample electrophoresis data to prepare a set of result data corresponding to the second set of sample electrophoresis data; and
display result data corresponding to the second set of sample electrophoresis data, the displayed result data including an indicium of whether a sample polynucleotide of the second set of sample electrophoresis data includes a mutation.

7. The system of claim 1, wherein the processor is configured to at least:
receive at least a second set of reference electrophoresis data;
receive user input defining members of a first group of sets of sample electrophoresis data for processing with respect to the first set of reference electrophoresis data, the first group of sets including the first set of sample electrophoresis data;
receive user input defining members of a second group of sets of sample electrophoresis for processing with respect to the second set of reference electrophoresis data;
process at least a subset of each member of the first group and at least a subset of the first set of reference electrophoresis data to prepare respective sets of result data corresponding to members of the first group; and
process at least a subset of each member of the second group and at least a subset of the second set of reference electrophoresis data to prepare respective sets of result data corresponding to members of the second group.

8. The system of claim 7, wherein the system includes a display and the processor is configured to simultaneously display result data corresponding to members of the first and second groups of sets of sample electrophoresis data.

9. The system of claim 8, wherein the displayed result data includes plurality of indicia, each of the indicia indicative of whether a sample polynucleotide of a respective one of the sets of sample electrophoresis data includes a mutation.

10. The system of claim 1, wherein the processor is configured to normalize at least one subset of the first set of sample electrophoresis data and the subset of the first reference electrophoresis data.

11. The system of claim 1, wherein the processor is configured to receive user input indicative of a size of the subset of the first set of sample electrophoresis data and a size of the subset of the first set of reference electrophoresis data and to process the subsets of electrophoresis data to prepare the result data corresponding to the first set of sample electrophoresis data.

12. The system of claim 1, further comprising a capillary array electrophoresis system configured to obtain sample electrophoresis data and reference electrophoresis data.

13. The system of claim 1, wherein the processor is configured to write the result data to a storage medium.

14. A computer-readable medium comprising executable software code, the code for determining the presence of a mutation in at least one sample polynucleotide, comprising:
code to receive (1) at least a first set of sample electrophoresis data indicative of a temperature gradient electrophoresis (TGE) separation of at least one sample polynucleotide, and (2) at least a first set of reference electrophoresis data indicative of a TGE separation of at least one reference polynucleotide; and
code to process at least a subset of the first set of sample electrophoresis data and at least a subset of the first set of reference electrophoresis data to prepare a set of result data corresponding to the first set of sample electrophoresis data, wherein the set of result data includes data indicative of whether a sample polynucleotide of the first set of sample electrophoresis data includes a mutation.

15. The computer-readable medium comprising executable software code of claim 14, wherein the code includes code to display result data corresponding to the first set of sample electrophoresis data.

16. The computer-readable medium comprising executable software code of claim 15, wherein the code includes code to display at least one indicium of whether a sample polynucleotide of the first set of sample electrophoresis data includes a mutation.

17. The computer-readable medium comprising executable software code of claim 16, wherein the code includes code to display an indicium of whether the first set of sample electrophoresis data includes more than one sample polynucleotide.

18. The computer-readable medium comprising executable software code of claim 17, wherein the code includes code to display an indicium corresponding to each sample polynucleotide of the first set of sample electrophoresis data, each indicium being indicative of whether a respective one of the sample polynucleotides includes a mutation.

19. The computer-readable medium comprising executable software code of claim 16, comprising:
code to receive at least a second set of sample electrophoresis data;
code to process at least a subset of the second set of sample electrophoresis data to prepare a set of result data corresponding to the second set of sample electrophoresis data; and
code to display result data corresponding to the second set of sample electrophoresis data, the displayed result data including an indicium of whether a sample polynucleotide of the second set of sample electrophoresis data includes a mutation.

20. The computer-readable medium comprising executable software code of claim 14, comprising:
code to receive at least a second set of reference electrophoresis data;
code to receive user input defining members of a first group of sets of sample electrophoresis for processing with respect to the first set of reference electrophoresis data, the first group of sets including the first set of sample electrophoresis data;
code to receive user input defining members of a second group of sets of sample electrophoresis for processing with respect to the second set of reference electrophoresis data;
code to process at least a subset of each member of the first group and at least a subset of the first set of reference electrophoresis data to prepare respective sets of result data corresponding to members of the first group; and
code to process at least a subset of each member of the second group and at least a subset of the second set of reference electrophoresis data to prepare respective sets of result data corresponding to members of the second group.

21. The computer-readable medium comprising executable software code of claim 20, comprising code to simultaneously display result data corresponding to members of the first and second groups of sets of sample electrophoresis data.

22. The computer-readable medium comprising executable software code of claim 21, comprising code to display a plurality of indicia, each of the indicia indicative of whether a sample polynucleotide of a respective one of the sets of sample electrophoresis data includes a mutation.

23. The computer-readable medium comprising executable software code of claim 14, comprising code to normalize at least one of the subset of the first set of sample electrophoresis data and the subset of the first reference electrophoresis data and to process the normalized data.

24. The computer-readable medium comprising executable software code of claim 14, comprising code to receive user input indicative of a size of the subset of the first set of sample electrophoresis data and a size of the subset of the first set of reference electrophoresis data and to process the subsets of electrophoresis data to prepare the result data corresponding to the first set of sample electrophoresis data.

25. A method for interacting with a computer to determine the presence of mutation in at least one sample polynucleotide, comprising the steps of:
executing an application which includes one or more windows having one or more controls;
manipulating at least one of said controls to define members of a group of sample electrophoresis data;
manipulating at least one of said controls for defining a set of reference electrophoresis data to be used for processing members of the group;
manipulating at least one of said controls to select a first subset of each member of the group and a first subset of the reference electrophoresis data; and
manipulating at least one of said controls to execute code configured to process the first subsets of the members of the group and the first subset of the set of reference electrophoresis data to prepare result data corresponding to each member of the group, wherein the result data corresponding to each member of the group includes data indicative of whether a first sample polynucleotide of the corresponding sample electrophoresis data includes a mutation.

26. The method of claim 25, further comprising the steps of:
manipulating at least one of said controls to select a second subset of each member of the group and a second subset of the reference electrophoresis data; and
manipulating at least one of said controls to execute code configured to process the second subsets of the members of the group and the second subset of the set of reference electrophoresis data to prepare result data corresponding to each member of the group, wherein the result data corresponding to each member of the group includes data indicative of whether a second sample polynucleotide of the corresponding sample electrophoresis data includes a mutation.

26. A method of processing electrophoresis data obtained by temperature gradient electrophoresis (TGE) separation to provide data indicative of the presence of a single polynucleotide polymorphism (SNP) or a mutation in a sample compound of a biological sample, comprising:
providing reference electrophoresis data comprising a plurality of reference data points (drᵢ, mᵢ), the reference data points defining at least one reference peak and having been obtained by TGE of at least one reference compound, and where, for the ith reference data point, drᵢ is a detection value indicative of a detector signal and mᵢ is a migration coordinate;
providing sample electrophoresis data comprising a plurality of sample data points (dsⱼ, mⱼ), the sample data points defining at least one sample peak and having been obtained by TGE of at least one sample compound, and where, for the jth sample data point, dsj is a detection value indicative of a detector signal and mj is a migration coordinate;
normalizing the reference and sample electrophoresis data based on at least one detection value of the reference electrophoresis data and at least one detection value of the sample electrophoresis data;
determining a plurality of detection value differences Δ between the reference and sample electrophoresis data, where the kth detection value difference Δₖ is indicative of a difference between (1) the detection value drᵢ of the reference electrophoresis data point having the migration coordinate mᵢ and (2) the detection value dsⱼ of the sample electrophoresis data point having the migration coordinate mⱼ; and
wherein the plurality of detection value differences Δ are indicative of the presence of an SNP or mutation in the sample compound of the biological sample.

27. The method of claim 26, further comprising determining the presence of an SNP or mutation in the sample compound on the basis of the plurality of detection value differences Δ.

28. The method of claim 26, further comprising the steps of:
determining a number of peaks present in the reference electrophoresis data;
determining a number of peaks present in the sample electrophoresis data; and
determining the presence of an SNP or mutation in the sample compound of the biological sample and the basis of (1) the plurality of differences Δ and (2) the number of peaks present in the reference and sample electrophoresis data.

29. The method of claim 26, comprising the steps of:
determining a migration coordinate difference Δₘ between migration coordinates of the reference and sample peaks; and
wherein, the step of determining a plurality of detection value differences Δ, comprises determining detection value differences between detection values of reference and sample electrophoresis data points that have migration coordinates differing by an amount Δₘ.

30. The method of claim 26, comprising the steps of:
determining a migration coordinate difference Δₘ between migration coordinates of the reference and sample peaks; and
prior to determining a plurality of detection value differences Δ, aligning the migration coordinates of the reference and sample electrophoresis data on the basis of the migration coordinate difference Δₘ.

31. A method of processing electrophoresis data obtained by temperature gradient electrophoresis (TGE) separation to provide data indicative of the presence of a single polynucleotide polymorphism (SNP) or a mutation in a sample compound of a biological sample, comprising:
providing reference electrophoresis data comprising a plurality of reference data points (drᵢ, mᵢ), the reference data points defining at least one reference peak and having been obtained by TGE of at least one reference compound, and where, for the ith reference data point, dsᵢ is a detection value indicative of a detector signal and mᵢ is a migration coordinate;
providing sample electrophoresis data comprising a plurality of sample data points (sⱼ, mⱼ), the sample data points defining at least one sample peak and having been obtained by TGE of at least one sample compound, and where, for the jth sample data point, sⱼ is a detection value indicative of a detector signal and mj is a migration coordinate;
normalizing the reference and sample electrophoresis data based on at least one detection value of the reference electrophoresis data and at least one detection value of the sample electrophoresis data; and
determining a covariance between the reference and sample electrophoresis data, wherein the covariance is indicative of the presence of an SNP or mutation in the sample compound of the biological sample.
